(19) United States
(12) Patent Application Publication
(10) Pub. No.: US 2024/0087746 A1
(43) Pub. Date: Mar. 14, 2024

(54) MEDICAL DIAGNOSTIC IMAGING APPARATUS AND MEDICAL INFORMATION PROCESSING METHOD

(71) Applicant: Canon Medical Systems Corporation, Otawara-shi (JP)

(72) Inventor: Junichiro ARAOKA, Nasushiobara (JP)

(73) Assignee: Canon Medical Systems Corporation, Otawara-shi (JP)

(21) Appl. No.: 18/466,037

(22) Filed: Sep. 13, 2023

(30) Foreign Application Priority Data
Sep. 13, 2022 (JP) 2022-145520
Sep. 12, 2023 (JP) 2023-147870

(51) Int. Cl. G16H 50/20; G16H 10/60; G16H 30/20; G16H 30/40; G16H 50/70

(52) U.S. Cl. CPC G16H 50/20; G16H 10/60; G16H 30/20; G16H 30/40; G16H 50/70

(57) ABSTRACT

According to one embodiment, a medical diagnostic imaging apparatus includes processing circuitry. The processing circuitry receives a subject's examination information including a case and an examination body part. The processing circuitry acquires a first search result by searching a library that associates an imaging protocol with each case using the subject's case. The processing circuitry acquires a second search result by searching a library that associates a slice condition with each examination body part using the subject's examination body part. The processing circuitry determines an imaging protocol for the subject based on the first search result, and determines a slice condition for the subject based on the second search result. The processing circuitry stores the received examination information and the determined imaging protocol and slice condition in a predetermined library in such a manner as to associate them with each other.

### CROSS-REFERENCE TO RELATED APPLICATIONS

This application is based upon and claims the benefit of priority from Japanese Patent Applications No. 2022-145520, filed Sep. 13, 2022; and No. 2023-147870, filed Sep. 12, 2023; the entire contents of all of which are incorporated herein by reference.

### FIELD

Embodiments described herein relate generally to a medical diagnostic imaging apparatus and a medical information processing method.

### BACKGROUND

A medical diagnostic imaging apparatus determines imaging conditions (e.g., an imaging protocol and a slice condition) appropriate for imaging a patient according to the examination information (e.g., the case and the examination body part) of the patient. Thus, a medical diagnostic imaging apparatus uses, for example, a conversion database which associates an appropriate combination of an imaging protocol and a slice condition with each combination of a case and an examination body part. Specifically, a medical diagnostic imaging apparatus determines imaging conditions appropriate for a patient by searching the conversion database using the examination information of the patient.

However, with regard to the conversion database described above, if there are many combinations of a case and an examination body part, there are also many combinations of an imaging protocol and a slice condition that are prepared. In this case, the amount of data managed by the conversion database increases. Consequently, a medical diagnostic imaging apparatus will, for example, need considerable time to search the conversion database and have difficulty in smoothly determining imaging conditions.

Especially in recent years, there has been a case where a medical diagnostic imaging apparatus further determines post-processing of a medical image (e.g., image processing), in addition to an imaging protocol and a slice condition, according to the examination information of a patient. There has also been a case where a medical diagnostic imaging apparatus determines an imaging protocol and a slice condition according to the personal information (e.g., gender, age group, weight) of a patient in addition to the examination information of the patient. Both of these cases particularly increase the amount of data managed by the conversion database. Therefore, it is desired to support determination of imaging conditions performed by a medical diagnostic imaging apparatus.

### BRIEF DESCRIPTION OF THE DRAWINGS

FIG. 1 is a block diagram showing an example of a configuration of a medical information processing system according to a first embodiment.

FIG. 2 is a block diagram showing an example of a configuration of a medical diagnostic imaging apparatus according to the first embodiment.

FIG. 3A is a diagram showing an example of an imaging protocol library according to the first embodiment.

FIG. 3B is a diagram showing an example of a slice condition library according to the first embodiment.

FIG. 3C is a diagram showing an example of a post-processing library according to the first embodiment.

FIG. 4 is a flow diagram showing an example of an operation of the medical diagnostic imaging apparatus according to the first embodiment.

FIG. 5 is a diagram showing an example of a selection menu of imaging protocols according to the first embodiment.

FIG. 6 is a diagram showing an example of an associating library according to the first embodiment.

FIG. 7 is a block diagram showing an example of a configuration of a medical information processing system according to a second embodiment.

FIG. 8 is a diagram showing an example of a detailed-condition library according to the second embodiment.

FIG. 9 is a flow diagram showing an example of an operation of a medical diagnostic imaging apparatus according to the second embodiment.

FIG. 10 is a diagram showing an example of a selection menu of imaging protocols according to the second embodiment.

### DETAILED DESCRIPTION

In general, according to one embodiment, a medical diagnostic imaging apparatus includes processing circuitry. The processing circuitry receives examination information including a case and an examination body part of a subject. The processing circuitry acquires a first search result by searching a first library that associates an imaging protocol with each case using the case of the subject as a first search key. The processing circuitry acquires a second search result by searching the first library that associates a slice condition with each examination body part or a second library that does not include the imaging protocol using the examination body part of the subject as a second search key. The processing circuitry determines an imaging protocol for the subject based on the first search result. The processing circuitry determines a slice condition for the subject based on the second search result. The processing circuitry stores the received examination information of the subject and the determined imaging protocol and slice condition in a predetermined library in such a manner as to associate them with each other.

Hereinafter, a medical diagnostic imaging apparatus and a medical information processing method according to embodiments will be described with reference to the accompanying drawings. In the embodiments described below, elements assigned with the same reference numerals perform the same operations, and redundant descriptions will be omitted as appropriate.

### First Embodiment

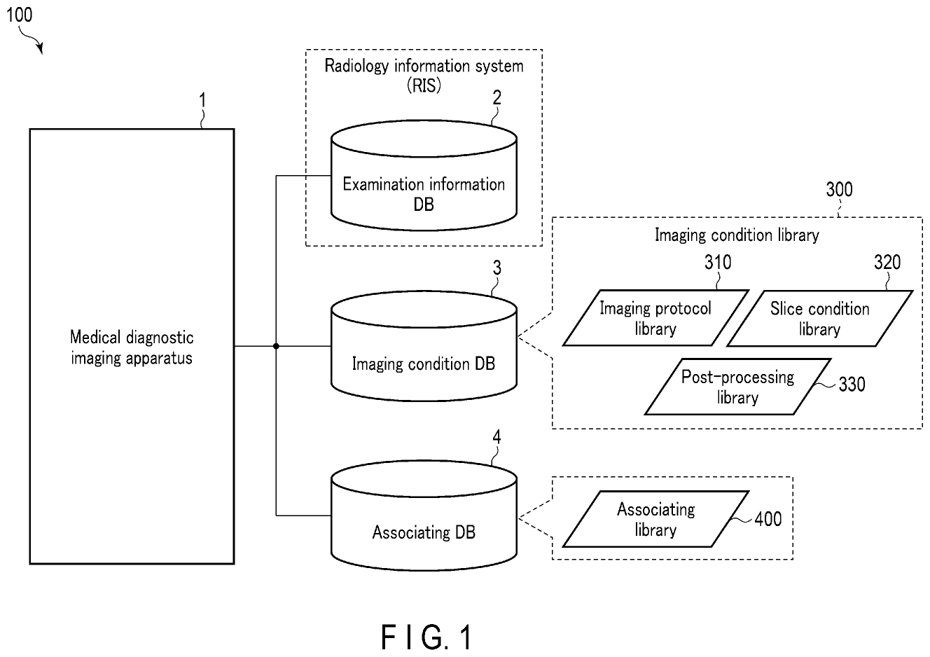

FIG. 1 is a block diagram showing an example of a configuration of a medical information processing system 100 according to a first embodiment. The medical information processing system 100 is a system of processing information relating to medical care, and includes a medical diagnostic imaging apparatus 1, an examination information DB 2, an imaging condition DB 3, and an associating DB 4. The medical diagnostic imaging apparatus 1 is connected to the examination information DB 2, the imaging condition DB 3, and the associating DB 4 so as to be able to communicate with them. At least one of the examination information DB 2, the imaging condition DB 3, or the associating DB 4 may be included in the medical diagnostic imaging apparatus 1.

The medical diagnostic imaging apparatus 1 is an apparatus that acquires a medical image for diagnosis of a patient. The medical diagnostic imaging apparatus 1 is, for example, an endoscope apparatus, a plain X-ray imaging apparatus, an X-ray computed tomography (CT) apparatus, a single photon emission computed tomography (SPECT) apparatus, a positron-emission tomography (PET) apparatus, a diagnostic magnetic resonance (MR) apparatus, a diagnostic ultrasonic (UL) apparatus, and an apparatus that combines these apparatuses (e.g., a SPECT-CT apparatus, a PET-CT apparatus). Hereinafter, a “patient” is an example of a subject. That is, each process according to the embodiment is also applicable to a subject other than a patient.

The examination information DB 2 is a database that stores the examination information (e.g., a case and an examination body part) of a patient. The examination information DB 2 is installed in, for example, a radiology information system (RIS). The examination information DB 2 may store an examination order issued by the radiology information system. The examination information DB 2 is an example of a storage.

The imaging condition DB 3 is a database that stores various imaging conditions (e.g., an imaging protocol, a slice condition, and post-processing). The imaging condition DB 3 includes an imaging protocol library 310, a slice condition library 320, and a post-processing library 330 as data of imaging conditions in the form of a library (hereinafter also referred to as “an imaging condition library 300”). The imaging condition DB 3 is an example of a storage.

The associating DB 4 is a database that stores the examination information of a patient and the imaging conditions determined relating to the patient in such a manner as to associate them with each other. The associating DB 4 includes an associating library 400 as data of examination information and imaging conditions in the form of a library. The associating DB 4 is an example of a storage.

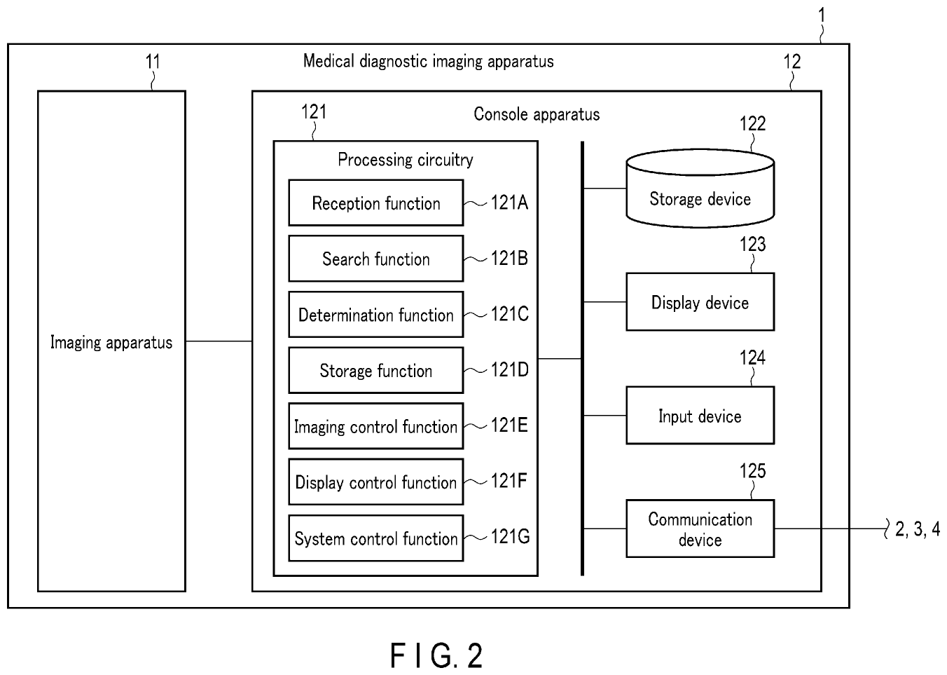

FIG. 2 is a block diagram showing an example of a configuration of the medical diagnostic imaging apparatus 1 according to the first embodiment. The medical diagnostic imaging apparatus 1 includes an imaging apparatus 11 and a console apparatus 12. The imaging apparatus 11 and the console apparatus 12 are connected to each other so as to be able to communicate with each other.

The imaging apparatus 11 is an apparatus that images a patient under predetermined imaging conditions. Specifically, the imaging apparatus 11 acquires a medical image of a patient by imaging the patient under a predetermined imaging protocol and a predetermined slice condition. The imaging apparatus 11 may further perform predetermined post-processing (e.g., image processing) on the medical image acquired. The imaging apparatus 11 transmits the medical image to the console apparatus 12. The imaging apparatus 11 is an example of an imaging unit.

The imaging apparatus 11 may acquire only imaging data (e.g., projection data) of a patient by imaging the patient under predetermined imaging conditions. In this case, the imaging apparatus 11 may transmit the acquired imaging data to the console apparatus 12. The console apparatus 12 may perform an image generating process or various types of image processing on the transmitted imaging data. That is, through collaboration between the imaging apparatus 11 and the console apparatus 12, a medical image of a patient may be acquired or various types of post-processing may be performed on the medical image.

The console apparatus 12 is an apparatus that controls the entire operation of the medical diagnostic imaging apparatus 1 and is a computer such as a workstation. Since the console apparatus 12 is an apparatus that processes information relating to medical care, it is also referred to as a “medical information processing apparatus”. The console apparatus 12 includes processing circuitry 121, a storage device 122, a display device 123, an input device 124, and a communication device 125.

The processing circuitry 121 is circuitry that controls the entire operation of the console apparatus 12 and includes at least one processor. The term “processor” means, for example, circuitry such as a CPU (central processing unit), a GPU (graphics processing unit), an ASIC (application specific integrated circuit), or a programmable logic device (for example, an SPLD (simple programmable logic device), a CPLD (complex programmable logic device), or an FPGA (field programmable gate array)), etc. If the processor is a CPU, the CPU implements each function by reading and executing the program stored in the storage device 122. If the processor is an ASIC, each function is directly incorporated into the circuitry of the ASIC as logic circuitry. The processor may be constituted in the form of single circuitry or in the form of multiple independent sets of circuitry that are combined. The processing circuitry 121 implements, for example, a reception function 121A, a search function 121B, a determination function 121C, a storage function 121D, an imaging control function 121E, a display control function 121F, and a system control function 121G.

The reception function 121A receives examination information including a case and an examination body part of a patient. Specifically, the reception function 121A receives examination information of a patient from the examination information DB 2 by using a modality worklist management (MWM) protocol of DICOM communication. The reception function 121A may store the received examination information in the storage device 122. At this time, the reception function 121A may perform line break control and trimming on the received examination information, and store the examination information that was subjected to these processes in the storage device 122. The reception function 121A is an example of a receiving unit. The receiving unit may also receive various kinds of information (e.g., gender, age group, weight) for the acquired examination information via the input device 124.

The search function 121B searches the imaging condition DB 3 using the examination information of the patient received by the reception function 121A as a search key. Firstly, the search function 121B searches the imaging protocol library 310 that associates an appropriate imaging protocol with each case using the case of the patient as a search key, and thereby acquires a search result. Secondly, the search function 121B searches the slice condition library 320 that associates an appropriate slice condition with each examination body part using the examination body part of the patient as a search key, and thereby acquires a search result. Thirdly, the search function 121B searches the post-processing library 330 that associates appropriate post-processing with each case using the case of the patient as a search key, and thereby acquires a search result. The “search result” includes the name and the total number (hit count) of the items that have matched a predetermined search key. The search function 121B may store the acquired search result in the storage device 122. The search function 121B is an example of a search unit.

The determination function 121C determines imaging conditions appropriate for the patient based on the search result acquired by the search function 121B. Firstly, the determination function 121C determines an imaging protocol appropriate for the patient based on the search result of the imaging protocol library 310. Secondly, the determination function 121C determines a slice condition appropriate for the patient based on the search result of the slice condition library 320. Thirdly, the determination function 121C determines post-processing appropriate for the patient based on the search result of the post-processing library 330. For example, if there is one search result, the determination function 121C determines imaging conditions corresponding to this search result as the imaging conditions appropriate for the patient. On the other hand, if there are “two or more” search results, the determination function 121C may cause the display control function 121F to display a selection menu for allowing an operator to select one search result from the multiple search results. The determination function 121C may store the determined imaging conditions in the storage device 122. The determination function 121C is an example of a determination unit.

The storage function 121D stores various kinds of data and information in the storage device 122 or the like. For example, the storage function 121D stores the examination information of the patient received by the reception function 121A and the imaging conditions of the patient determined by the determination function 121C in the associating library 400 of the associating DB 4 in such as manner as to associate them with each other. The storage function 121D is an example of a storage.

The imaging control function 121E controls imaging of the patient performed by the imaging apparatus 11. Specifically, the imaging control function 121E controls the imaging apparatus 11 based on the imaging conditions determined by the determination function 121C, and thereby acquires a medical image of the patient. The imaging control function 121E may store the medical image acquired in the storage device 122. The imaging control function 121E is an example of an imaging controller.

The display control function 121F causes various images to be displayed on the display device 123. Firstly, if the search result of the imaging protocol library 310 acquired by the search function 121B includes multiple imaging protocol candidates, the display control function 121F causes a selection menu for allowing an operator to select one among the multiple imaging protocol candidates to be displayed on the display device 123. Secondly, if the search result of the slice condition library 320 acquired by the search function 121B includes multiple slice condition candidates, the display control function 121F causes a selection menu for allowing an operator to select one among the multiple slice condition candidates to be displayed on the display device 123. Thirdly, if the search result of the post-processing library 330 acquired by the search function 121B includes multiple post-processing candidates, the display control function 121F causes a selection menu for allowing an operator to select one among the multiple post-processing candidates to be displayed on the display device 123. That is, the display control function 121F generates display data of the selection menu and outputs the generated display data to the display device 123. The display control function 121F is an example of a display controller.

The display control function 121F may display the medical image of the patient acquired by the imaging control function 121E. The display control function 121F may also display an operation screen for an operator to control the operation of the console apparatus 12. The operator may acquire the examination information of the patient by inputting a predetermined operation on this operation screen via the input device 124 or register various kinds of information (e.g., gender, age group, weight) for the acquired examination information.

The system control function 121G controls the entire operation of the console apparatus 12. The system control function 121G is an example of a system controller.

The storage device 122 is a device that stores various kinds of data and information. For example, the storage device 122 is a processor-readable storage medium (e.g., a magnetic storage medium, an electromagnetic storage medium, an optical storage medium, a semiconductor memory). Alternatively, the storage device 122 may be a drive that reads and writes various kinds of data and information from and to the storage medium. The storage device 122 may include at least one of the examination information DB 2, the imaging condition DB 3, or the associating DB 4. The storage device 122 is an example of a storage.

The display device 123 is a device for displaying various kinds of data and information. For example, the display device 123 is a liquid crystal display, a plasma display, an organic EL (electro-luminescence) display, or an LED display. In particular, the display device 123 may be a touch-panel display that also serves as the input device 124. The display device 123 is an example of a display unit.

The input device 124 is a device for receiving various types of input from an operator. Specifically, the input device 124 converts various types of input received from an operator into electric signals, and transmits the electric signals to the processing circuitry 121. Examples of the input device 124 include a mouse, a keyboard, a button, a panel switch, a slider switch, a trackball, an operation panel, and a touch panel. The input device 124 is an example of an input unit.

The communication device 125 is a device that communicates with the examination information DB 2, the imaging condition DB 3, and the associating DB 4 to exchange various kinds of data and information with them. The DICOM protocol or HL7 protocol may be applied to this communication. The communication device 125 is an example of a communication unit.

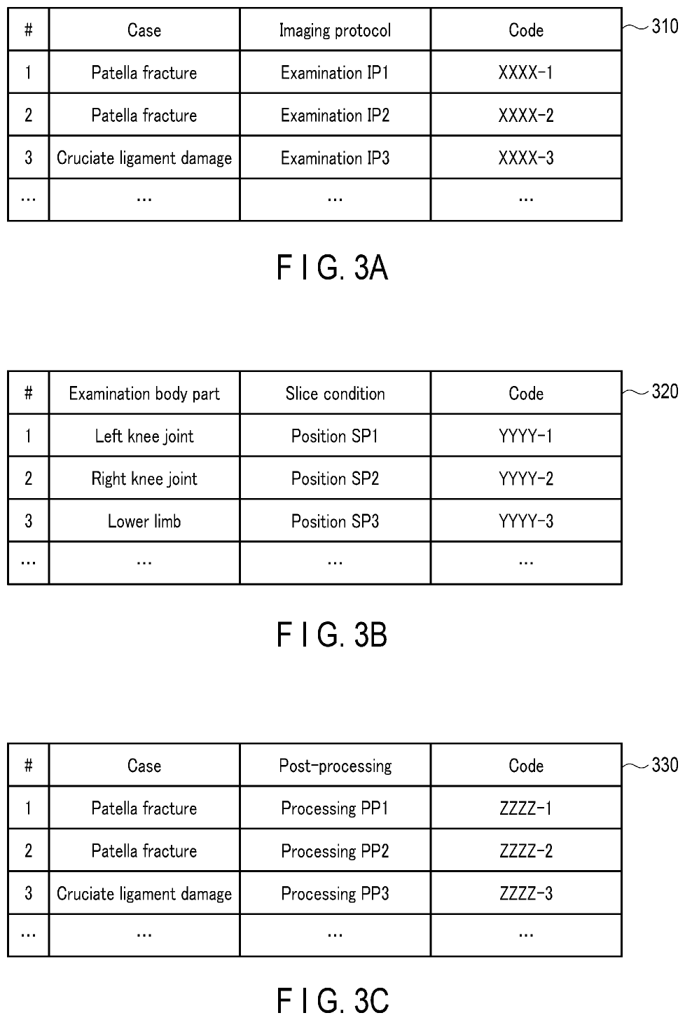

FIG. 3A is a diagram showing an example of the imaging protocol library 310 according to the first embodiment. In the imaging protocol library 310, an appropriate imaging protocol and a code uniquely specifying the imaging protocol are associated with each case. For example, in the first record (#1), a case “patella fracture”, an imaging protocol “examination IP1”, and a code “XXXX-1” are associated with one another.

In the embodiment, the “imaging protocol” includes a set of defined scan parameters used for predetermined examination. A plurality of imaging protocols may be associated with the same case. For example, multiple imaging protocols (e.g., examination X for leptosome, examination X for standard type, examination X for pyknic type) varying by the body type (e.g., leptosome, standard type, pyknic type) of the patient are associated with the same case.

FIG. 3B is a diagram showing an example of the slice condition library 320 according to the first embodiment. In the slice condition library 320, an appropriate slice condition and a code uniquely specifying the slice condition are associated with each examination body part. For example, in the first record (#1), an examination body part “left knee joint”, a slice condition “position SP1”, and a code “YYYY-1” are associated with one another.

In the embodiment, the “slice condition” includes the plane direction (angle), the thickness, and the number of slices used in a predetermined examination. The slice condition may be a coordinate in a three-dimensional orthogonal coordinate system.

FIG. 3C is a diagram showing an example of the post-processing library 330 according to the first embodiment. In the post-processing library 330, appropriate post-processing and a code uniquely specifying the post-processing are associated with each case. For example, in the first record (#1), a case “patella fracture”, post-processing “process PP1”, and a code “ZZZZ-1” are associated with one another.

In the embodiment, the “post-processing” includes types of post-processing (e.g., a multi-planar reconstruction (MPR) method, difference processing) and various parameters (e.g., an MPR optimum cross-section, a window width (WW), a window level (WL), a weighting coefficient).

The “codes” stored in the imaging protocol library 310, the slice condition library 320, and the post-processing library 330 may be given according to a predetermined rule. For example, if the examination body part includes a left or right identification flag, as in “left” or “right” (e.g., left knee joint, right knee joint), a code indicating “left” may include “L”, and a code indicating “right” may include “R”. Also, to each small examination body part (e.g., a mouth, a nose, an ear, a brain) included in a large examination body part (e.g., a head region), a code corresponding to the large examination body part may be given uniformly. This allows reduction of the amount of data managed by the imaging protocol library 310, the slice condition library 320, and the post-processing library 330.

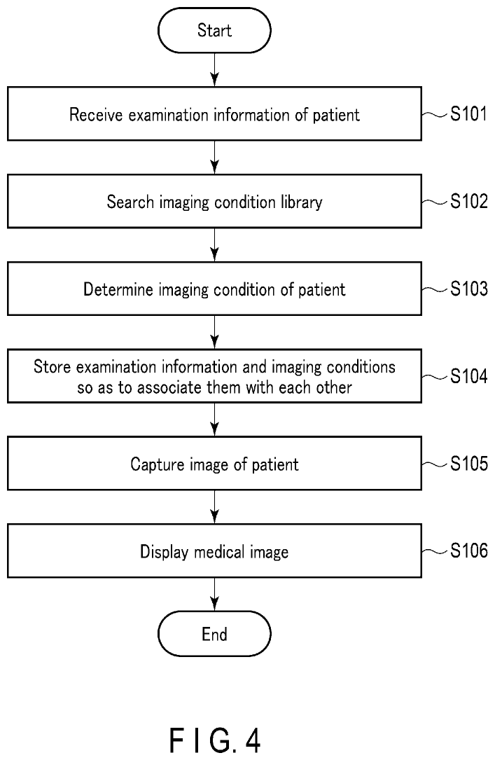

FIG. 4 is a flow diagram showing an example of an operation of the medical diagnostic imaging apparatus 1 according to the first embodiment. This exemplary operation may be started when an operator inputs a start instruction via the input device 124.

(Step S101) First, the medical diagnostic imaging apparatus 1 receives the examination information of a patient with the reception function 121A. Specifically, the reception function 121A receives the examination information of a patient from the examination information DB 2.

(Step S102) Next, the medical diagnostic imaging apparatus 1 searches the imaging condition library 300 with the search function 121B. Specifically, the search function 121B extracts a case and an examination body part of the patient from the examination information of the patient received in step S101. Firstly, the search function 121B searches the imaging protocol library 310 using the extracted case of the patient as a search key, and thereby acquires a search result. Secondly, the search function 121B searches the slice condition library 320 using the extracted examination body part of the patient as a search key, and thereby acquires a search result. Thirdly, the search function 121B searches the post-processing library 330 using the extracted case of the patient as a search key, and thereby acquires a search result.

(Step S103) Subsequently, the medical diagnostic imaging apparatus 1 determines the imaging conditions of the patient with the determination function 121C. Specifically, the determination function 121C determines the imaging conditions of the patient based on the search result acquired in step S102. In particular, if the search result acquired in step S102 includes multiple imaging condition candidates, the display control function 121F may cause a selection menu for allowing the operator to select one among the multiple imaging condition candidates to be displayed on the display device 123.

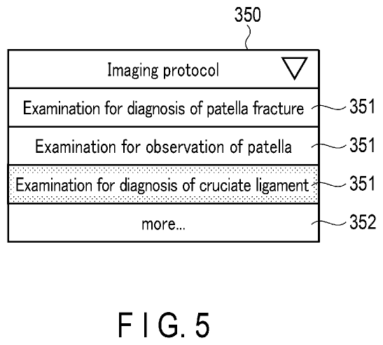

FIG. 5 is a diagram showing an example of a selection menu 350 of imaging protocols according to the first embodiment. The selection menu 350 includes multiple items 351 respectively corresponding to the multiple imaging protocol candidates. In particular, a button 352 for displaying all the imaging protocol candidates is adjacent to the item 351 located in the lower stage of the selection menu 350.

The operator may select a desired item 351 on the selection menu 350 via the input device 124. In this example, the item 351 corresponding to “examination for diagnosis of cruciate ligament” is selected. In this case, the determination function 121C may determine the “examination for diagnosis of cruciate ligament” as the imaging protocol of the patient.

(Step S104) The explanation continues with reference back to FIG. 4. Subsequently, with the storage function 121D, the medical diagnostic imaging apparatus 1 stores the examination information and the imaging conditions in such a manner as to associate them with each other. Specifically, the storage function 121D stores the examination information of the patient received in step S101 and the imaging conditions of the patient determined in step S103 in the associating library 400 included in the associating DB 4 in such a manner as to associate them with each other. The storage function 121D may store the data associating the examination information and the imaging conditions in the examination information DB 2.

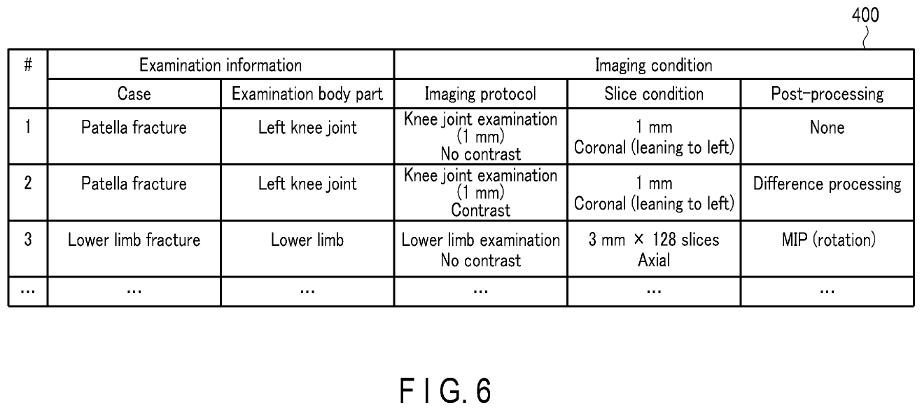

FIG. 6 is a diagram showing an example of the associating library 400 according to the first embodiment. In the associating library 400, each piece of the examination information of the patient is associated with the imaging conditions determined for the examination information. For example, in the first record (#1), the examination information “Case: patella fracture, Examination body part: left knee joint” and the imaging conditions “Imaging protocol: knee joint examination (1 mm); no contrast, Slice condition: 1 mm; coronal (closer to left), Post-processing: none” are associated with each other.

In the associating library 400, up to N (N: natural number) imaging conditions may be stored for predetermined examination information. If a new imaging condition is to be stored for predetermined examination information, an old imaging condition that has been stored for this predetermined examination information may be deleted. The date and time when the association mentioned in step S104 was performed may also be stored in the associating library 400. Namely, the associating library 400 holds a history of the past association.

Needless to say, imaging condition candidates that are limited on the basis of the associating library 400 may be presented to the operator. For example, the medical diagnostic imaging apparatus 1 searches the associating library 400 for examination information that is the same as or similar to the examination information of the patient received. As a result of this search, the medical diagnostic imaging apparatus 1 may present, as an imaging condition appropriate for the patient, an imaging condition corresponding to specific examination information identified in the associating library 400. In other words, the medical diagnostic imaging apparatus 1 associates a specific record among the multiple records included in the associating library 400 with the patient. The medical diagnostic imaging apparatus 1 may store the date and time when this association was performed in association with the specific record.

(Step S105) The explanation continues with reference back to FIG. 4. Subsequently, the medical diagnostic imaging apparatus 1 acquires an image of the patient with the imaging control function 121E. Specifically, the imaging control function 121E acquires an image of the patient based on the imaging conditions determined in step S103. For example, the imaging control function 121E acquires an image of the patient based on the imaging protocol and the slice condition determined in step S103. The imaging control function 121E processes the medical image of the patient in the post-processing determined in step S103. Through this processing, a post-processed image of the patient is acquired.

(Step S106) Finally, the medical diagnostic imaging apparatus 1 displays the medical image with the display control function 121F. Specifically, the display control function 121F causes the medical image of the patient acquired in step S105 to be displayed on the display device 123. Steps S105 and S106 may be performed before step S104. After step S106, the medical diagnostic imaging apparatus 1 ends a series of processing.

Above are descriptions of the medical information processing system 100 according to the first embodiment. According to the first embodiment, the medical diagnostic imaging apparatus 1 determines the imaging conditions of the patient using the data of the imaging protocols, slice conditions, and post-processing (included in the imaging conditions) in the form of libraries (i.e., the imaging protocol library 310, the slice condition library 320, and the post-processing library 330). That is, the medical diagnostic imaging apparatus 1 uses multiple libraries that multi-dimensionally manage the imaging conditions instead of using one conversion database that one-dimensionally manages the imaging conditions. Since the pieces of information contained in a conversion database are dispersed in multiple libraries, the medical diagnostic imaging apparatus 1 can quickly search for necessary information, as compared to the case of using the conversion database. Therefore, the medical diagnostic imaging apparatus 1 can smoothly determine the imaging conditions of a patient.

In general, a “case” of a patient is associated with an “imaging protocol” and “post-processing”, and an “examination body part” of a patient is associated with a “slice condition”. Thus, if the case of a patient is the same as the previous one when the medical diagnostic imaging apparatus 1 acquires an image of the patient this time, the same imaging protocol and post-processing as the previous ones may be applied this time. On the other hand, if the “examination body part” of the patient is the same as the previous one, the same slice condition as the previous one may be applied this time. Thus, dispersing the pieces of information contained in a conversion database in multiple libraries based on this assumption can reduce the amount of data managed by the medical diagnostic imaging apparatus 1 and reduce the chance of managing duplicate data due to combinations of a case and an examination body part. As a result, the maintenance of the medical diagnostic imaging apparatus 1 is improved

### Second Embodiment

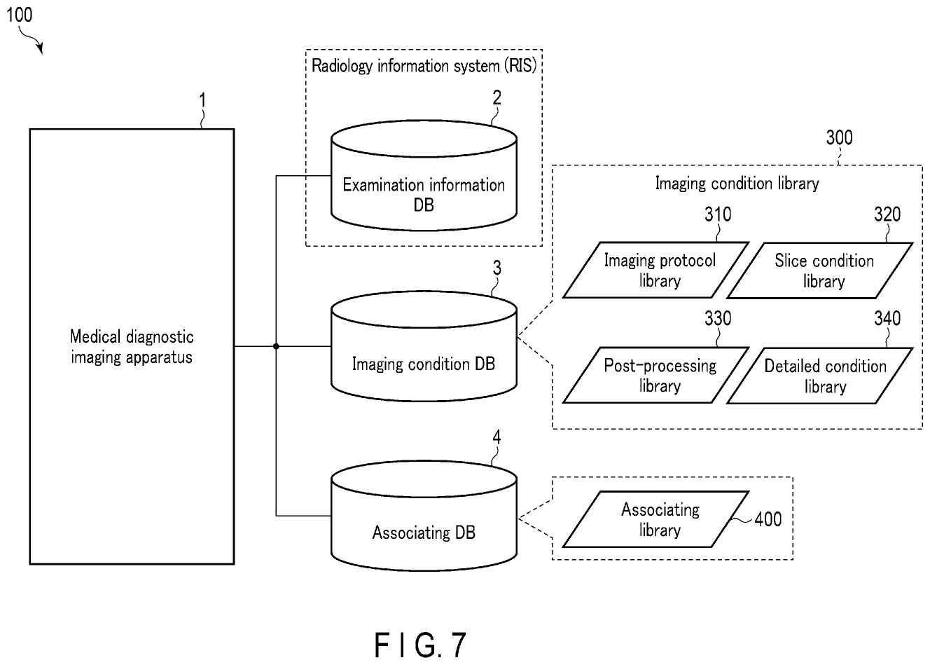

FIG. 7 is a block diagram showing an example of a configuration of a medical information processing system 100 according to a second embodiment. Unlike the first embodiment, in the second embodiment, the examination information DB 2 includes personal information of a patient (e.g., gender, age group, weight, presence or absence of an allergy, pulse (i.e., pulse rate, information relating to the presence or absence of arrhythmia, etc.), and cardiorespiratory function (i.e., how long an examination subject can hold breath, information relating to the presence or absence of abnormal respiration, etc.)). These kinds of personal information may be input from various measuring devices (for example, the information on a pulse may be input from a pulse monitor), or input by an operator's hand. In addition, the imaging condition DB 3 includes a detailed-condition library 340 as the imaging condition library 300. The other aspects of the second embodiment are the same as what is described in the first embodiment.

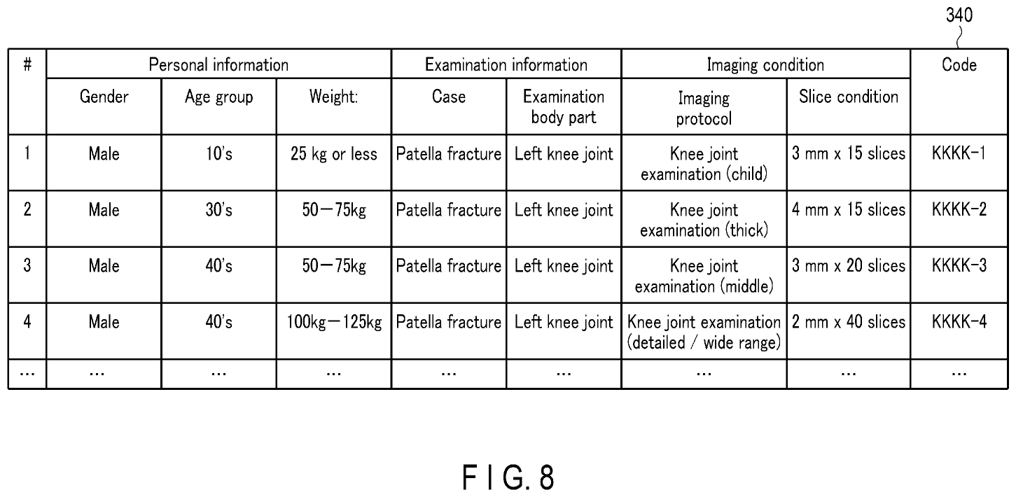

FIG. 8 is a diagram showing an example of the detailed-condition library 340 according to the second embodiment. In the detailed-condition library 340, appropriate imaging conditions and a code uniquely specifying the imaging conditions are associated with each piece of personal information and each piece of examination information. For example, in the first record (#1), the personal information “Gender: male, Age group: 10's, Weight: 25 kg or less”, the examination information “Case: patella fracture, Examination body part: left knee joint”, the imaging conditions “Imaging protocol: knee joint examination (child), Slice condition: 3 mm×15 slices”, and the code “KKKK-1” are associated with each other.

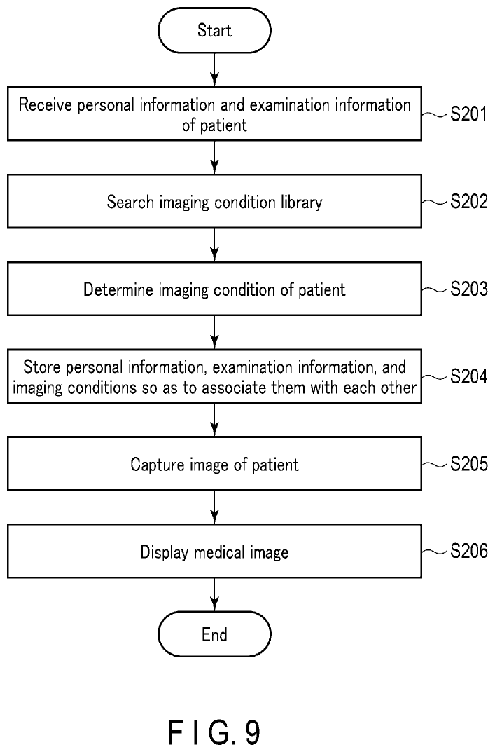

FIG. 9 is a flow diagram showing an example of an operation of the medical diagnostic imaging apparatus 1 according to the second embodiment. As in the case of the exemplary operation shown in FIG. 4, the exemplary operation shown in FIG. 9 may be started when an operator inputs a start instruction via the input device 124.

(Step S201) First, the medical diagnostic imaging apparatus 1 receives the personal information and examination information of a patient with the reception function 121A. Specifically, the reception function 121A receives the personal information and examination information of a patient from the examination information DB 2.

(Step S202) Next, the medical diagnostic imaging apparatus 1 searches the imaging condition library 300 with the search function 121B. Specifically, the search function 121B searches the detailed-condition library 340 included in the imaging condition DB 3 using the personal information and the examination information of the patient received in step S201 as a search key, and thereby acquires a search result. Needless to say, the search function 121B also performs similar processing to that described in the first embodiment.

In this instance, a case where the personal information of the patient is “Gender: male, Age group, 20's, Weight: 50 to 75 kg” is assumed. If the detailed-condition library 340 does not have a record that includes the same personal information as this personal information, the search function 121B may, instead, search for a record that includes personal information similar to this personal information. Then, the search function 121B may extract, as imaging condition candidates of the patient, the imaging conditions included in the record searched for.

Alternatively, the search function 121B may calculate a similarity between the personal information of the patient and the personal information of each record included in the detailed-condition library 340. Then, the search function 121B may extract the imaging conditions included in a record whose similarity satisfies a predetermined threshold as imaging condition candidates of the patient. Needless to say, the search function 121B may extract, as candidates, the imaging conditions included in multiple records whose similarity is within the top N.

Let us also assume the case where the search function 121B searches the detailed-condition library 340 using the “weight” of the patient as a search key and where the received examination information does not include the “weight” of the patient. In this case, the search function 121B may start searching the detailed-condition library 340 after the operator inputs the weight of the patient via the input device 124.

(Step S203) Subsequently, the medical diagnostic imaging apparatus 1 determines the imaging conditions of the patient with the determination function 121C. Specifically, the determination function 121C determines the imaging conditions of the patient based on the search result acquired in step S202. In particular, if the search result acquired in step S202 includes multiple imaging condition candidates, the display control function 121F may cause a selection menu for allowing the operator to select one among the multiple imaging condition candidates to be displayed on the display device 123.

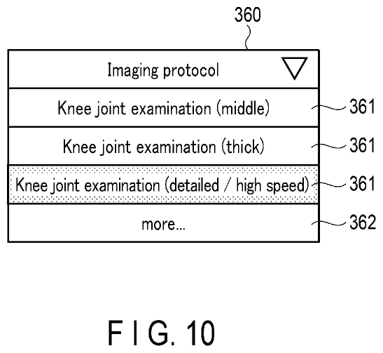

FIG. 10 is a diagram showing an example of a selection menu 360 of imaging protocols according to the second embodiment. The selection menu 360 shown in FIG. 10, like the one shown in FIG. 5, includes multiple items 361 respectively corresponding to the multiple imaging protocol candidates. In particular, a button 362 for displaying all the imaging protocol candidates is adjacent to the item 361 located in the lower stage of the selection menu 360.

The operator may select a desired item 361 on the selection menu 360 via the input device 124. In this example, the item 361 corresponding to “knee joint examination (detailed/high speed)” is selected. In this case, the determination function 121C may determine “knee joint examination (detailed/high speed)” as the imaging protocol of the patient.

(Step S204) The explanation continues with reference back to FIG. 9. Subsequently, with the storage function 121D, the medical diagnostic imaging apparatus 1 stores the personal information, the examination information, and the imaging conditions in such a manner as to associate them with each other. Specifically, the storage function 121D stores the personal information and the examination information of the patient received in step S201 and the imaging conditions of the patient determined in step S203 in the associating library 400 included in the associating DB 4 in such a manner as to associate them with each other. The storage function 121D may store the data associating the patient's information, the examination information, and the imaging conditions in the examination information DB 2.

(Step S205) Subsequently, the medical diagnostic imaging apparatus 1 acquires an image of the patient with the imaging control function 121E. Step S205 is similar to step S105.

(Step S206) Finally, the medical diagnostic imaging apparatus 1 displays the medical image with the display control function 121F. Step S206 is similar to step S106.

Above are descriptions of the medical information processing system 100 according to the second embodiment. According to the second embodiment, the medical diagnostic imaging apparatus 1 determines the imaging conditions of the patient using data of the imaging conditions in the form of a library for each piece of personal information and each piece of examination information (i.e., the detailed-condition library 340). Thus, the medical diagnostic imaging apparatus 1 can determine imaging conditions appropriate for each patient. The medical diagnostic imaging apparatus 1 can also present plausible imaging conditions under complicated conditions. In addition, since the associating library 400 of the associating DB 4 is updated every time an operator uses the medical diagnostic imaging apparatus 1, the medical information processing system 100 can save an operator or the like the trouble of preparing a conversion database having a large amount of data.

(Modifications)

A modification of the medical information processing system 100 according to the first and second embodiments will be described below. Firstly, the imaging condition library 300 need not be revised on a daily basis. For example, the imaging condition library 300 is revised in such cases as (A) updating the medical diagnostic imaging apparatus 1, (B) optimizing parameters by adding optional features, (C) adjusting image quality by changing doctors who interpret radiogram, and (D) reassessing the examination to be performed due to addition of a diagnosis-treatment department, a change in the allocation of equipment, or the like.

Secondly, an “operation mode” may be set in the medical diagnostic imaging apparatus 1. For example, in an “operation mode II”, the medical diagnostic imaging apparatus 1 may use a case and an examination body part of a patient to determine an imaging protocol and a slice condition appropriate for the patient. On the other hand, in an “operation mode I”, the medical diagnostic imaging apparatus 1 uses an examination body part of a patient to determine a slice condition appropriate for the patient but does not determine an imaging protocol. Instead, the medical diagnostic imaging apparatus 1 causes a selection menu of imaging protocols to be displayed on the display device 123 and allows an operator to select one imaging protocol on the selection menu. On the other hand, in an “operation mode X”, the medical diagnostic imaging apparatus 1 does not determine an imaging protocol or a slice condition that are appropriate for the patient.

Thirdly, a “select mode” may be set in the medical diagnostic imaging apparatus 1. For example, in a “select mode II”, the medical diagnostic imaging apparatus 1 displays a selection menu of imaging conditions. On the other hand, in a “select mode I”, the medical diagnostic imaging apparatus 1 selects one imaging condition from multiple imaging conditions by majority vote. Also, in a “select mode X”, the medical diagnostic imaging apparatus 1 selects an imaging condition lastly selected by an operator.

Fourthly, the imaging protocol, the slice condition, the post-processing, and the personal information are not necessarily managed in individual libraries, respectively; that is, multiple kinds of information may be managed in the same library. In this case, for example, the imaging protocol is managed in the imaging protocol library 310, and the slice condition, the post-processing, and the personal information are managed in a common library different from the imaging protocol library 310.

Fifthly, the detailed-condition library 340 may include an “examination posture” as the “examination information” in addition to the “case” and the “examination body part”. In this case, appropriate “imaging conditions” are associated with each case, each examination body part, and each examination posture in the detailed-condition library 340.

The examination posture is a posture of a patient subjected to examination, and is kinematically expressed by a combination of a “body position” and a “pose” of the patient. The body position indicates a positional relation of the body with the direction of gravitational force, and is, for example, a standing position, a recumbent position (face-up position, lateral recumbent position, face-down position), or a seated position. The pose indicates a relative positional relation among body parts, and is, for example, a position of 0° flexion of a shoulder joint, a position of 180° flexion of a shoulder joint, a position of 0° flexion of a knee joint, or a position of 90° flexion of a knee joint.

Needless to say, the examination posture may be expressed qualitatively, as in “a state in which an upper limb is stretched”, “a state in which an upper limb is bent”, “a state in which a lower limb is stretched”, or “a state in which a lower limb is bent”.

The medical diagnostic imaging apparatus 1 acquires a search result by searching the detailed-condition library 340 using, as a search key, the personal information of a patient and the examination information including the examination posture of the patient. The medical diagnostic imaging apparatus 1 determines imaging conditions appropriate for the patient based on the acquired search result. That is, the medical diagnostic imaging apparatus 1 can determine imaging conditions appropriate for the patient in more detail based further on the examination posture of the patient.

According to at least one of the embodiments described above, determination of imaging conditions can be supported.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical diagnostic imaging apparatus comprising processing circuitry configured to:
receive examination information including a case and an examination body part of a subject;
acquire a first search result by searching a first library using the case of the subject as a first search key, the first library associating an imaging protocol with each case;
acquire a second search result by searching the first library that associates a slice condition with each examination body part or a second library that does not include the imaging protocol using the examination body part of the subject as a second search key and;
determine an imaging protocol for the subject based on the first search result;
determine a slice condition for the subject based on the second search result; and
store the received examination information of the subject and the determined imaging protocol and slice condition in a predetermined library in such a manner as to associate them with each other.

2. The medical diagnostic imaging apparatus according to claim 1, wherein if the first search result includes multiple imaging protocol candidates, the processing circuitry causes a selection menu for allowing an operator to select one among the multiple imaging protocol candidates to be displayed on a display.

3. The medical diagnostic imaging apparatus according to claim 1, wherein the processing circuitry is configured to:
acquire a third search result by searching a third library that associates post-processing with each case using the case of the subject as a third search key;
determine post-processing for the subject based on the third search result; and
store the received examination information of the subject and the determined post-processing in a predetermined library in such a manner as to associate them with each other.

4. The medical diagnostic imaging apparatus according to claim 3, further comprising an imaging device configured to acquire an image of the subject,
wherein the processing circuitry is configured to acquire a medical of the subject by controlling the imaging device based on the determined imaging protocol, slice condition, and post-processing.

5. The medical diagnostic imaging apparatus according to claim 1, wherein the processing circuitry is configured to:
further receive personal information including at least one of a gender, an age group, a weight, a presence or absence of an allergy, a pulse, or a cardiorespiratory function of the subject;
acquire a fourth search result by searching a fourth library that associates an imaging protocol and a slice condition with each piece of personal information and each piece of examination information using the personal information and the examination information of the subject as a fourth search key, and
determine an imaging protocol and a slice condition for the subject based on the fourth search result.

6. The medical diagnostic imaging apparatus according to claim 1, wherein
the examination information of the subject further includes an examination posture of the subject, and
the processing circuitry is configured to:
further receive personal information including at least one of a gender, an age group, a weight, a presence or absence of an allergy, a pulse, or a cardiorespiratory function of the subject;
acquire a fourth search result by searching a fourth library that associates an imaging protocol and a slice condition with each piece of personal information and each piece of examination information using the personal information and the examination information of the subject as a fourth search key, and
determine an imaging protocol and a slice condition for the subject based on the fourth search result.

7. A medical information processing method comprising:
receiving examination information including a case and an examination body part of a subject;
acquiring a first search result by searching a first library using the case of the subject as a first search key, the first library associating an imaging protocol with each case;
acquiring a second search result by searching the first library that associates a slice condition with each examination body part or a second library that does not include the imaging protocol using the examination body part of the subject as a second search key and;
determining an imaging protocol for the subject based on the first search result;
determining a slice condition for the subject based on the second search result; and
storing the received examination information of the subject and the determined imaging protocol and slice condition in a predetermined library in such a manner as to associate them with each other.
